(19) United States
(12) Patent Application Publication
(10) Pub. No.: US 2025/0377345 A1
(43) Pub. Date: Dec. 11, 2025

(54) Flue Gas Analysis Device

(71) Applicant: Seitron S.P.A., Mussolente (VI) (IT)

(72) Inventor: Carlo Scoccia, Bassano del Grappa (IT)

(21) Appl. No.: 18/734,941

(22) Filed: Jun. 5, 2024

(51) Int. Cl. G01N 33/00

(52) U.S. Cl. CPC G01N 33/0068; G01N 33/004; G01N 33/0032

(57) ABSTRACT

A flue gas analysis device includes a display; an inlet port for the flue gases; a first sensor configured to detect the level of a first chemical species, selected from oxygen and carbon dioxide, present in the flue gases and to generate as output first data signals associated with the level of the first detected chemical species; and a logic unit operatively connected to the display and configured to receive the first data signals as input. The logic unit is configured to electronically control the display so as to: display a stoichiometric combustion diagram including a first axis indicating excess air; display a first indicator along the first axis of the excess air value calculated by the logic unit by processing the first data signals; and dynamically vary the position of the first indicator along the first axis according to the instantaneous value of the first data signals.

### CROSS-REFERENCE TO RELATED APPLICATIONS

Not applicable.

### BACKGROUND OF THE DISCLOSURE

### 1. The Field of the Disclosure

The invention relates to a device for analyzing flue gases. In particular, a device for analyzing flue gases coming from a boiler.

### 2. The Relevant Technology

Gas boilers normally use fuel, typically natural gas, to generate heat. These boilers are commonly used for both domestic heating and industrial applications.

Gas is sent to the burner inside the boiler. The burner is the component that burns gas to generate heat. There are different types of burners, but all of them have the purpose of providing a flame that heats water or generates steam.

The heat produced by the burner flame is transferred to the water by means of a heat exchanger. This heat exchanger is designed to efficiently transfer the combustion-generated heat to the water or fluid to be heated.

Gases generated by combustion inside the boiler are channeled through a combustion chamber and then directed to a discharge device, usually a chimney or an evacuation duct.

It is crucial for combustion to take place efficiently in the burner in order to obtain energy savings. By an efficient combustion, a greater amount of energy is extracted from the fuel used. This results in less fuel consumption to produce the same amount of heat, thus leading to energy cost savings.

Furthermore, an efficient combustion is necessary to reduce emissions from polluting agents as an inefficient combustion with a lack of oxygen can lead to the formation of carbon monoxide (CO) and can generate a greater amount of pollutant emissions such as nitrogen oxides (NOₓ), sulfur dioxide (SO₂) and particulate matter.

In addition to this, inefficient combustion can cause increased stress on boiler components, reducing lifetime thereof and increasing the need for maintenance and repairs. Efficient combustion, on the other hand, can help extend the life of the boiler and reduce maintenance costs.

In normal gas boilers, the fuel is natural gas, the main component of which is methane (CH₄), which combines with the comburent, i.e., oxygen (O₂).

Methane gas combustion can be complete, the most efficient, or incomplete, the least efficient. The latter occurs when there is a lack of oxygen, i.e., when not enough air has been introduced into the combustion chamber, leading to the formation of carbon monoxide (CO) and other by-products.

Complete combustion occurs when enough oxygen is present in the combustion chamber to burn all the methane gas. This reaction produces carbon dioxide (CO₂) and water vapor (H₂O).

The introduction of the right amount of air into the combustion chamber is, therefore, crucial to obtain complete combustion.

Combustion efficiency is closely linked to the correct use of the amount of oxygen required to consume the fuel. Too little oxygen can lead to incomplete combustion, while too much oxygen can cause a loss of energy through excessive heating of the combustion air.

Maximum efficiency is obtained with stoichiometric combustion, which represents an ideal theoretical condition wherein the optimum amount of oxygen and fuel generates the maximum amount of heat possible, thus achieving maximum combustion efficiency.

However, in practice, it is difficult to reach this ideal condition due to variations in operating conditions and fuel characteristics. Thus, in combustion optimization, it is important to accurately balance the amount of oxygen and fuel to get as close as possible to stoichiometric combustion, thus maximizing the efficiency of the combustion process.

The measurement of combustion efficiency in boilers is usually done by flue gas analysis.

Flue gas analysis devices are known to detect the presence and amounts of carbon monoxide, carbon dioxide and oxygen in these gases.

Depending on the type of boiler, there will be a high efficiency range for each of these parameters that can be achieved by adjusting the air entering the combustion chamber.

Flue gas analysis devices can have available a digital display on which indicator bars are visible, each indicating the value of one of the measured components (O₂, CO, CO₂).

Thus, the operator in charge of installing or maintaining the boiler, by means of this flue gas analysis device, sees the measured values and, by adjusting the air to the combustion chamber, tries to place such values in high efficiency ranges.

However, reading these indicator bars is not very intuitive for the operator.

The presence of a bar for each measured value, while providing a useful indication, may be difficult to read for an inexperienced operator.

In fact, one of the major difficulties in reading the instrument and consequently adjusting the air to the combustion chamber is that the operator may not easily understand whether more or less air is required.

### SUMMARY OF THE DISCLOSURE

The task of the present invention is to develop a device for analyzing flue gases that can overcome the aforementioned drawbacks and limitations of the prior art.

In particular, the object of the present invention is to make a device whose use is more intuitive for an operator.

Yet another object of the invention is to develop a device that makes it easier for the operator to understand whether more or less air is required.

One or more the above-mentioned tasks and objects are achieved by a flue gas analysis device disclosed herein.

Further characteristics of the device are described in the claims.

### BRIEF DESCRIPTION OF THE DRAWINGS

The aforesaid task and objects, together with the advantages that will be mentioned hereinafter, are indicated by the description of an embodiment of the invention, which is given by way of non-limiting example with reference to the attached drawings, where:

FIG. 1 represents a perspective view of a device according to the invention;

FIG. 2 represents the device of FIG. 1 in an off configuration;

FIG. 3 represents a front view of the device of FIG. 1;

FIG. 4 represents a detail of the device of FIG. 3 at a possible time of measurement,

FIG. 5 represents the detail of FIG. 4 at a further possible time of measurement.

### DETAILED DESCRIPTION OF THE PREFERRED EMBODIMENTS

With reference to the above-mentioned figures, a device for analyzing flue gases according to the invention is globally referred to as 10.

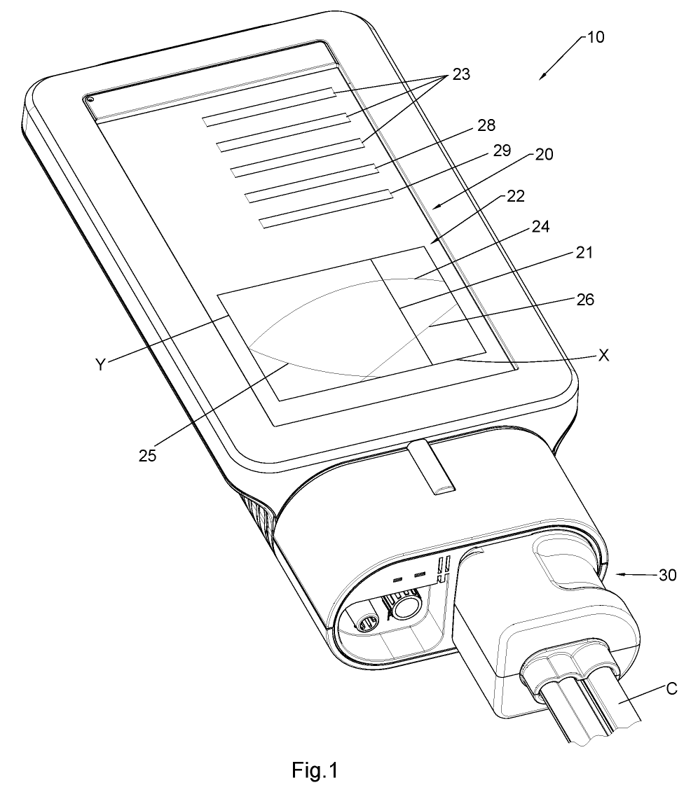

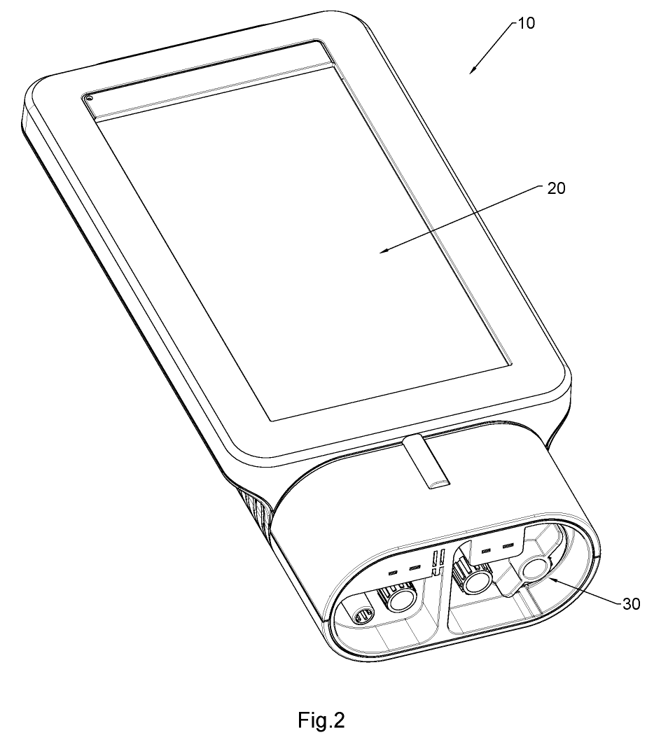

Such a device 10, clearly visible in FIGS. 1, 2 and 3, comprises:

a display 20;

an inlet port 30 for flue gases;

a first sensor configured to detect the level of a first chemical species in the flue gases and to generate as output first data signals associated with the level of that first detected chemical species;

a logic unit operatively connected to the display 20 and configured to receive these first data signals as input.

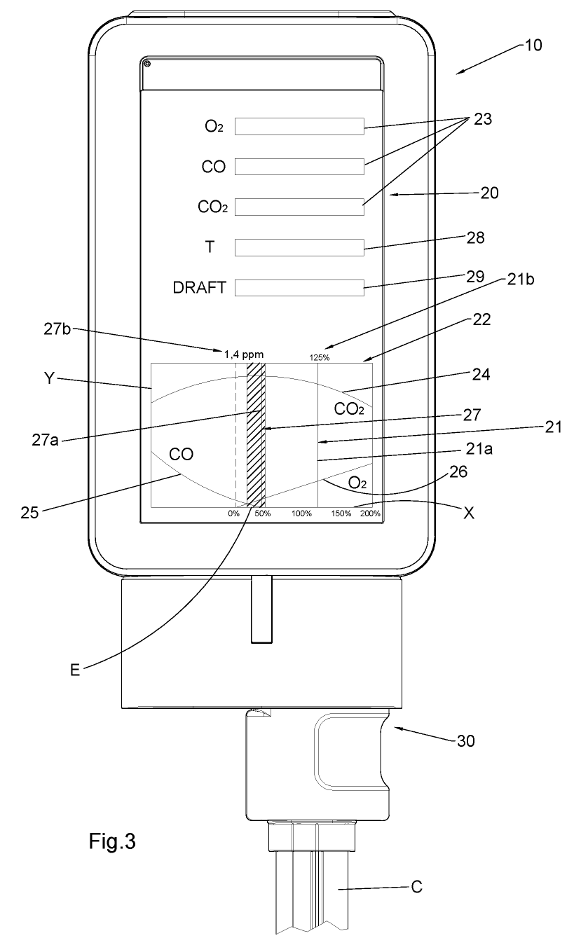

In FIGS. 1 and 3, the inlet port 30 is connected to a duct C for gases coming from a boiler.

According to the invention, this first chemical species is oxygen or carbon dioxide.

It is worth emphasizing that the first sensor can detect either oxygen or carbon dioxide because, as known, the value of the former can be obtained from the latter, and vice versa.

The peculiarity of the device 10 lies in the fact that the logic unit is configured to electronically control the display 20 so as to display a stoichiometric combustion diagram 22, clearly visible in FIGS. 1 and 3.

A stoichiometric combustion diagram 22 refers to a known-type diagram wherein a first axis X indicates the excess air.

Excess air refers to the greater amount of air present in the fuel mixture compared to the theoretical amount of air required for a complete combustion.

It is known how to calculate excess air from the level of oxygen or carbon dioxide.

In particular, the excess air, which can be referred to as e, is calculated by means of the air index, which can be referred to as n, through the formula e=n-1.

The air index n is defined as n=21/(21-O₂ ) or as n=CO₂ₜ /CO₂ whereby O₂ denotes the detected oxygen level, CO₂ denotes the level of detected carbon dioxide and CO₂ₜ denotes a constant that varies according to the fuel used. Such constant CO₂ₜ amounts to:

11.7 in case the fuel is natural gas,

13.9 in case the fuel is propane or LPG or butane,

15.1 in case the fuel is diesel,

15.7 in case the fuel is fuel oil.

It is therefore once again clear how the first sensor can detect either oxygen or carbon dioxide.

In the preferred embodiment of the invention, the excess air is indicated as a percentage of the amount of air needed to achieve stoichiometric combustion.

For example 0% indicates that there is no excess air and that combustion is theoretically stoichiometric.

The logic unit is configured to electronically control the display 20 so as to display an indicator 21 along the first axis X of the excess air value calculated by the aforesaid logic unit by processing the first data signals.

This indicator 21 thus indicates the excess air value in the combustion chamber.

The logic unit is configured to process the first and second data signals and calculate this excess air value.

In addition, the logic unit is configured to electronically control the display 20 so as to dynamically vary the position of the indicator 21 along the first axis X according to the instantaneous value of the first and second data signals.

While being adjusted, the indicator 21 will dynamically shift to zones of greater or lesser air excess depending on whether an operator varies the air flow to the combustion chamber allowing for a greater or lesser flow rate.

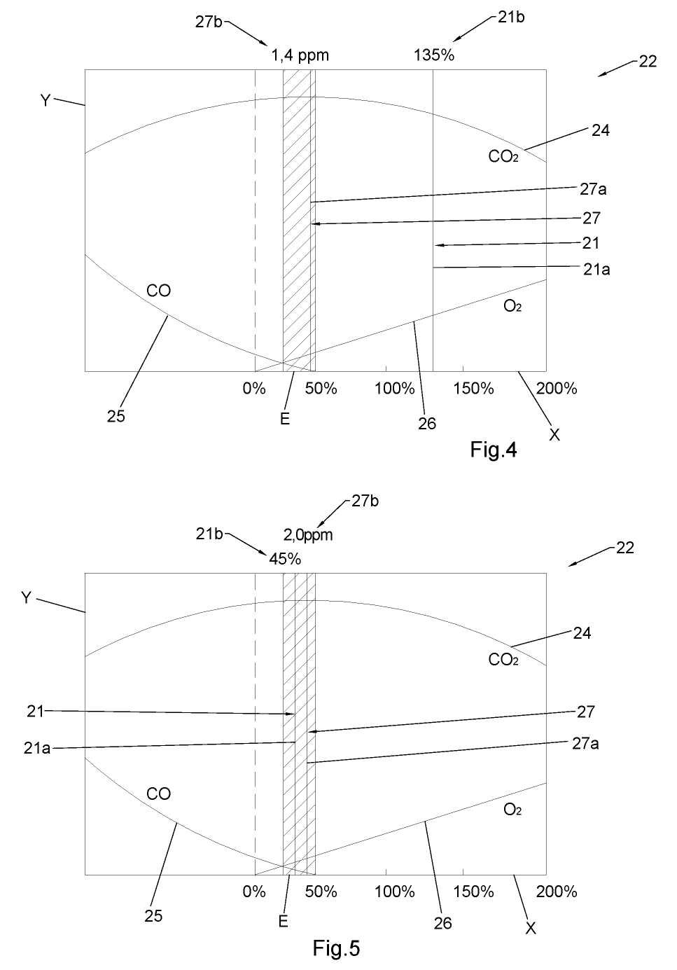

Examples are shown in FIGS. 4 and 5: in the former there is a high excess of air, in the latter there is a limited excess of air.

This configuration makes it possible to make a device that is more intuitive for an operator.

Furthermore, advantageously, an easy interpretation of the measured values is achieved.

The configuration described, by indicating the direction in which the involved variables will change, makes it easier for the operator to understand whether more or less air is required.

Advantageously, a qualitative indication of the excess air value is obtained, and this indication is easy to interpret by an operator in charge of maintaining or installing a boiler.

This is because having an indication of the excess air makes it easier for an operator to make air adjustments to the combustion chamber, in particular it is more intuitive to understand whether more or less air is required.

According to the invention, the logic unit is configured to electronically control the display 20 so as to display along the first axis X a range E of excess air values that leads to high boiler efficiency. The logic unit is configured to calculate this range E according to user-settable parameters.

A high efficiency range refers to a range defined on the axis X indicating the excess air and defining values consecutive to each other at which a high combustion efficiency is obtained.

Of course, the values at which high efficiency is achieved also depend on the type of boiler.

For example, user-settable parameters are accessible from a corresponding menu shown on the display 20.

The user will set these parameters according to the type of boiler.

The described configuration is particularly advantageous in that a user of the device 10, e.g., the technician in charge of installing or maintaining the boiler, by adjusting the air to the combustion chamber, tries to set the excess air within the high efficiency range E.

In order to achieve this adjustment, what is required is for the indicator 21 to be positioned within the high efficiency range E, as visible in FIG. 5.

Continuing the description of the preferred embodiment of the invention, the device 10 comprises a second sensor configured to detect the level of a second chemical species, namely carbon monoxide, present in flue gases and to generate as output second data signals associated with the level of that second detected chemical species.

The logic unit is configured to receive such second data signals as input.

In addition, the logic unit is configured to electronically control the display 20 so as to display a second indicator 27 of the carbon monoxide level on the stoichiometric combustion diagram 22.

In addition, the logic unit is configured to dynamically vary the position of this second indicator 27 according to the instantaneous carbon monoxide level.

This configuration is particularly useful for displaying the measured carbon monoxide value directly on the stoichiometric combustion diagram 22.

Furthermore, one may wonder whether this second indicator 27 is redundant with the first excess air indicator 21. However, it is worth emphasizing that the stoichiometric combustion diagram 22 is a theoretical diagram, while combustion in the combustion chamber and the gases produced by the latter may slightly deviate from the theoretical value predicted by the diagram. Thus, the first and second indicators 21, 27, although connected by the physics of actual combustion, provide the operator with two substantially different but both fundamental pieces of information: one related to combustion efficiency and the other related to the carbon monoxide content measured in the exhaust gases. Consequently, they are not redundant with respect to each other.

The stoichiometric combustion diagram 22 is a diagram known in the art, however, it will be explained more precisely in the herein description.

The stoichiometric combustion diagram 22 comprises at least one trend line 24, 25, 26 of one of these chemical species as a function of excess air.

Preferably, the stoichiometric combustion diagram 22 comprises a first line 24 of the carbon monoxide CO trend as a function of excess air.

Preferably, the stoichiometric combustion diagram 22 comprises a second line 25 of the carbon dioxide CO₂ trend as a function of excess air.

Preferably, the stoichiometric combustion diagram 22 comprises a third line 26 of the oxygen O₂ trend as a function of excess air.

In addition, the stoichiometric combustion diagram 22 comprises a second axis Y indicating the amount of chemical species, e.g., oxygen, carbon monoxide or carbon dioxide.

In the preferred embodiment of the invention, the second axis Y does not show a scale indicating the amount of chemical species, as the diagram provides a more qualitative indication.

However, it must not be ruled out that, in a possible version of the invention, the second axis Y shows a scale indicating the amount of chemical species.

Moreover, the presence of at least one of these trend lines 24, 25, 26 in combination with the indicator 21 allows the operator to read from the diagram a qualitative indication of the measured amount of this chemical species.

In a possible version of the invention, the stoichiometric combustion diagram 22 comprises an additional efficiency line as a function of excess air.

Returning to the description of the preferred embodiment of the invention, the logic unit is configured to electronically control the display 20 so as to display at least one indicator bar 23 indicating at least one of the levels of such chemical species.

Preferably, the display 20 is configured to show three indicator bars 23 indicating oxygen, carbon dioxide and carbon monoxide levels.

In addition, the logic unit is configured to electronically control the display 20 so as to dynamically vary such at least one indicator bar 23 according to the data signals received as input.

It is worth emphasizing that the stoichiometric combustion diagram 22 in combination with the indicator 21 provides more qualitative information.

The indicator bars, on the other hand, 23 provide a more quantitative indication.

The configuration described thus provides complete information on the parameters measured during the flue gas analysis.

Returning to the description of the preferred embodiment of the invention, the first indicator 21 comprises a segment 21a perpendicular to the first axis X.

In addition, the second indicator 27 comprises a second segment 27a perpendicular to the first axis X.

Such a configuration makes it easier to read the stoichiometric combustion diagram 22 and the measured values.

According to the invention, the first indicator 21 comprises digital numerical values 21b.

Such digital numerical values 21b indicate the calculated value of excess air.

As visible in FIGS. 3 to 5, the digital numerical values 21b are arranged above the segment 21a.

In addition, the second indicator 27, if present, may also comprise digital numeric values 27b.

Such digital numerical values 27b indicate the measured value of carbon monoxide.

As visible in FIGS. 3 to 5, the digital numerical values 27b are arranged above the segment 27a.

This configuration makes the reading of the device 10 easier by providing a qualitative and quantitative indication in the same diagram.

Returning to the description of the preferred embodiment of the invention, the device 10 comprises a third sensor configured to detect the temperature of the flue gas and generate as output third data signals associated with the measured temperature.

Obviously, the logic unit is configured to receive such third data signals as input, possibly process them and transmit a signal to the display 20.

In addition, the logic unit is configured to electronically control the display 20 so as to display an additional indicator bar 28 indicating the measured temperature.

The logic unit is configured to electronically control the display 20 so as to dynamically vary this additional indicator bar 28 based on the third data signals.

Finally, the logic unit is configured to calculate the aforementioned range E according to on these third data signals.

Specifically, the logic unit calculates the range E according to user-settable parameters and the third data signals.

As already mentioned previously, the range E is a range of excess air values that leads to high boiler efficiency.

The temperature, therefore, helps to define the values of excess air that lead to high boiler efficiency.

As known, knowledge of flue gas temperature is crucial in flue gas analysis for several reasons.

Furthermore, the flue gas temperature is an indicator of the overall efficiency of the heating system, with lower temperatures indicating lesser heat losses through the chimney and higher temperatures indicating greater energy dispersion with the flue gases.

Returning to the description of the preferred embodiment of the invention, the device 10 comprises a fourth sensor configured to detect the chimney draft and to generate as output fourth data signals associated with that draft.

The logic unit is configured to receive such fourth data signals as input, possibly process them and transmit a signal to the display 20.

In addition, the logic unit is configured to electronically control the display 20 so as to display an additional indicator bar 29 indicating this draft.

The logic unit is configured to electronically control the display 20 so as to dynamically vary this indicator bar 29 according to the fourth data signals.

It has in practice been established that the invention achieves the intended task and objects. In particular, with the invention, a device was developed that is more intuitive for an operator.

In addition, a device was developed to help the operator understand whether more or less air is required, in order to optimize the combustion process.

What is claimed is:

1. A flue gas analysis device, comprising:
a display;
an inlet port for said flue gases;
a first sensor configured to detect the level of a first chemical species, selected from oxygen and carbon dioxide, present in said flue gases and to generate as output first data signals associated with the level of said first detected chemical species;
a logic unit operatively connected to said display and configured to receive said first data signals as input;
wherein said logic unit is configured to electronically control said display so as to:
display a stoichiometric combustion diagram comprising a first axis indicating excess air;
display a first indicator along said first axis of the excess air value calculated by said logic unit by processing said first data signals;
dynamically vary the position of said first indicator along said first axis according to the instantaneous value of said first data signals.

2. The device according to claim 1, wherein the logic unit is configured to electronically control said display so as to display along said first axis a range of excess air that leads to high boiler efficiency, said logic unit being configured to calculate said range according to user-settable parameters.

3. The device according to claim 1, further comprising a second sensor configured to detect the level of a second chemical species present in said flue gases and to generate as output second data signals associated with the level of said second detected chemical species, said logic unit being configured to receive said second data signals as input.

4. The device according to claim 3, wherein the second chemical species is carbon monoxide and the logic unit is configured to electronically control said display so as to:
display on said stoichiometric combustion diagram a second indicator of the carbon monoxide level,
dynamically vary the position of said second indicator according to the instantaneous carbon monoxide level measured by said second sensor.

5. The device according to claim 3, wherein the stoichiometric combustion diagram comprises at least one trend line of one of said chemical species as a function of excess air.

6. The device according to claim 3, wherein the logic unit is configured to electronically control said display so as to display at least one indicator bar indicating at least one of said levels of chemical species.

7. The device according to claim 6, wherein the logic unit is configured to electronically control said display so as to dynamically vary said at least one indicator bar according to said first and/or second data signals.

8. The device according to claim 1, wherein the stoichiometric combustion diagram comprises an additional efficiency trend line as a function of the excess air.

9. The device according to claim 1, wherein the first indicator comprises a segment perpendicular to said first axis.

10. The device according to claim 1, wherein the first indicator comprises digital numerical values.

11. The device according to claim 2, further comprising a third sensor configured to detect the temperature of said flue gases and to generate as output third data signals associated with said temperature, said logic unit being configured to:
electronically control said display so as to display an additional indicator bar indicating said measured temperature; and
calculate said range according to said user-settable parameters and said third data signals.
